(19) United States
(12) Patent Application Publication
(10) Pub. No.: US 2015/0328240 A1
(43) Pub. Date: Nov. 19, 2015

(54) PRODUCT AND DELIVERY SYSTEM FOR APPLICATION OF ANTIMICROBIAL TREATMENT DESIGNED TO INHIBIT PATHEGENS FROM ENTERING OR LEAVING A RESPITORY SYSTEM AND TO REMOVE PATHOGENS FROM WOUNDS, EARS OR OTHER BODY CAVITIES, AND METHODS OF USE

(71) Applicants: Carl Hilliard, Del Mar, CA (US); William R. Cast, Fort Wayne, IN (US)

(72) Inventors: Carl Hilliard, Del Mar, CA (US); William R. Cast, Fort Wayne, IN (US)

(21) Appl. No.: 14/716,566

(22) Filed: May 19, 2015

Related U.S. Application Data
(60) Provisional application No. 62/000,403, filed on May 19, 2014.

(51) Int. Cl. A61K 31/695; A61L 31/16; A61K 45/06

(52) U.S. Cl. CPC A61K 31/695; A61K 45/06; A61L 31/16; A61L 2300/404; A61L 2300/622

(57) ABSTRACT

An antimicrobial product and methods of use are provided. The antimicrobial product includes a water-soluble antimicrobial organosilane and the product delivered in an alcohol, foam or gel state in some cases including various additional compounds including antibiotic and anti-inflammatory medications, antiseptics, and/or detergents. An example delivery system including a microcapsule encasing the product or a particle coated with the product is also described. Methods of use include embedding a delivery system within an article, the microcapsule containing the product which is released by a mechanism and at a time particular to the intended use of the article.

### CROSS REFERENCE TO RELATED APPLICATION

This application claims priority from U.S. Patent Application No. 62/000,403, filed May 19, 2014 and entitled “Antimicrobial Polymer Products and Delivery System for Infection Control And Method of Using The Same,” which is incorporated entirely herein by reference.

### BACKGROUND

1. Technical Field

This disclosure relates generally to antimicrobial compounds with antiseptic and antibiotic activity; in particular, to organosilane compounds for use against infectious pathogens that can be inhaled or expelled on or present in infected wounds or areas and and methods of using the same.

2. State of the Art

Prevention and treatment of infection in humans and animals has been a public health goal since the discovery of microorganisms and their role in causing disease. As an outgrowth of the germ theory of disease, much progress has been made in controlling the spread, dissemination, and effects of pathogenic microorganisms. For example, simple techniques such as routine hand-washing and thorough cleaning of hard surfaces are highly effective in preventing the spread of diseases which are disseminated by contact. One such method is by the use of face masks that are designed to filter out, and, in some cases, destroy microorganisms by use of toxic chemicals embedded in the mask. Some masks are used for a short duration, for example surgical masks. Other masks are worn by the general population for longer periods, usually when the wearer is ill for the duration of a respiratory infection. The disadvantages of these methods is that either live pathogens remain embedded in the used face mask or the mask contains chemicals that are harmful to the environment when disposed. Furthermore, the presence of toxic biocides in the environment tends to lead to mutations in microorganisms that then become resistant to such biocides. When infection occurs despite such precautions, treatment with topical and systemic antimicrobials, such as the use of antibiotics, are valuable adjuncts to these preventive measures. Another such method is by application of bandages and dressings that have been saturated with toxic biocides or antibiotics to infected areas. The same disadvantages to this treatment apply in that the pathogens mutate and the discarded hazardous waste poses a risk of harm to the environment in both the handling and disposal of the used product. WHO has called antimicrobial resistance is “so serious that it threatens the achievements of modern medicine.”

Microorganisms, including bacteria, viruses, and fungi, are present in the environment and can be spread from carrier to carrier over air currents or surface to surface or through physical contact. It is increasingly recognized that antibiotics developed to combat single cell (planktonic) microbes are becoming less and less effective. The medical response has been to prescribe higher dosage levels of antibiotics but with diminishing results. Another effect of the over use of antibiotics is that they leach into the environment. Compounds with lower or diminished levels of concentration have been discarded into the environment as waste allowing pathogens coming into contact with such diluted compounds to withstand their effect and become resistant to such antibiotics.

To address the exhaustion of biocides to the surrounding environment, a class of water-soluble antimicrobial polymers (Contact-Active Biocidal, “CAB”) was developed to provide a non-toxic, non-leaching surface covering. CAB products can be bonded to most surfaces, both porous and non-porous, for an extended period of time. As such, the CAB products provide an invisible microbiostatic coating that kills single cell microbes which drift onto the surface. The CAB products are typically offered in liquid form and may be applied to surfaces such as walls or counter tops after disinfecting the surface or on clothing through a washing machine rinse cycle. Once the CAB product is applied, the compound establishes a killing field for new microbes by creating a semi-permanent coating which mechanically kills microorganisms on contact.

The effective life of the CAB product, however, is relatively short. Moreover, once applied, it is difficult to determine at what time the biological activity becomes diminished and the CAB is no longer maintaining a disinfected surface. An undisclosed problem is a CAB not regularly cleaned can be expected to fill with dust and debris which creates an inviting Conditioning Layer and works counter to its claimed purpose. As a result, most CAB products used have not been a commercial success. Accordingly, there is a need for a different, novel and creative use for CAB compounds that address the concerns presented herein above; namely, providing an effective supplement for antibiotics in treating surface infections; inhibiting the transfer of microorganisms, body fluids and particulate matter; reducing use of toxic compounds that pollute the environment, and reduce the likelihood of treatment resistant strains which may spread and have deadly consequences.

### DISCLOSURE OF EMBODIMENTS OF THE INVENTION

The present disclosure relates to antimicrobial compounds, and, in particular, to organosilane-containing antimicrobial products and methods of use for treating microbial infections and reducing the speed of pathogens.

Disclosed is an antimicrobial product comprising an organosilane alone and together with other compounds; a carrier; and a delivery system.

In some embodiments, the delivery system is a microcapsule enclosing the antimicrobial product therein. In some embodiments, the delivery system is a tiny particle coated with the antimicrobial product. In some embodiments, the antimicrobial product further is accompanied by a fragrance, wherein under a condition wherein the microcapsule is broken, the antimicrobial product is adhered onto an article and the fragrance becomes active; and wherein a time of useful antimicrobial activity of the activated antimicrobial product corresponds to a time wherein a scent of the active fragrance is present. In some embodiments, the organosilane is a 3-(trimethoxysilyl) quaternary ammonium compound or a 3-(trihydroxysilyl) quaternary ammonium compound.

In some embodiments, the concentration of the organosilane is less than 0.10 percent by weight. In some embodiments, the concentration of the organosilane is between 0.10 percent and 1.00 percent by weight. In some embodiments, the concentration of the organosilane is greater than 1.00 percent by weight. In some embodiments, the concentration of the organosilane is greater than 5.0 percent by weight.

In some embodiments, the carrier is a compound selected from the group of carrier compounds consisting of an alcohol, a wax, or dimethylsulfoxide. In some embodiments, the antimicrobial product further utilizes an enzyme of bacterial origin, preferably from a Bacillus or Actinomyces, or from fungal sources or genetically engineered from non-alkaline cellulases by modifying the protein to function in an alkaline pH range. In some embodiments, the enzyme is a proteolytic keritinase enzyme. In some embodiments, the enzyme is an enzyme acting upon a substrate comprising N-acyl homoserine lactone. In some embodiments, the antimicrobial product further includes a detergent. In some embodiments, the detergent is a quaternary ammonium compound.

In some embodiments, the antimicrobial product further comprises a disinfectant. In some embodiments, the antimicrobial product further comprises a local anesthetic. In come embodiments, the local anesthetic is a compound selected from the group of local anesthetic compounds consisting of lidocaine hydrochloride, and benzocaine hydrochloride. In some embodiments, the antimicrobial product includes a buffer. In some embodiments, the buffer is a compound selected from the group of buffer compounds consisting of a citrate, a sulfonate, a carbonate, and a phosphate.

Disclosed is a method of providing a non-toxic antimicrobial treatment to a dressing, gauze, spunlace, nonwoven, melt blown or other materials, the method comprising the steps of applying a delivery system for a product containing an organosilane; activating the delivery system; and adhering the organosilane.

Disclosed is a method of providing a non-toxic antimicrobial treatment to dressings for site infections and filtering materials, the method comprising the steps of embedding a surface during manufacture of the surface of the item with a microcapsule enclosing an antimicrobial product comprising an organosilane and releasing the antimicrobial product from the capsule.

The foregoing and other features and advantages of the present invention will be apparent from the following more detailed description of the particular embodiments of the invention, as illustrated in the accompanying drawings.

### BRIEF DESCRIPTION OF THE DRAWINGS

Some of the embodiments will be described in detail, with reference to the following figures, wherein like designations denote like members:

FIG. 1 is a schematic diagram showing a general chemical structure of an organosilane molecule;

FIG. 2 is a schematic diagram showing a general chemical structure of an organosilane molecule;

FIG. 3 is a schematic representation showing organosilane molecules adhered to a dressing or filter in the presence of microbial cells;

FIG. 4 is a schematic representation of a delivery system comprising a microcapsule for an antimicrobial product;

FIG. 5 is a schematic representation of an article embedded with a delivery system for an antimicrobial product;

FIG. 6 is a schematic representation of a delivery system comprising microcapsules enclosing an antimicrobial product, an aging indicator, and a fragrance;

FIG. 7 is a diagram of a method 200 of treating infection and/or infectious disease and/or providing long-lasting disinfectant properties to a dressing or filter;

FIG. 8 is a diagram of a method 300 of treating infection and/or infectious disease and/or providing short term disinfectant properties to a dressing or filter;

FIG. 9 is a diagram of a method 400 of forming an article with an antimicrobial product delivery system embedded therein; and

FIG. 10 is a diagram of a method 500 of treating and preventing an infection on a filter.

### DETAILED DESCRIPTION OF EMBODIMENTS OF THE INVENTION

A detailed description of the hereinafter described embodiments of the disclosed apparatus and method are presented by way of example and not meant to be limiting with reference to the Figures listed above. Although certain embodiments are shown and described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure.

As a preface to the detailed description, it should be noted that, as used in this specification and the appended claims, the singular forms “a”, “an” and “the” include plural referents, unless the context clearly dictates otherwise. Some general definitions are provided for the terms used herein. “Organosilane” means a compound of the family of compounds comprising the elements of silicon, oxygen, and carbon with a C—Si covalent bond and a nitrogen atom in a quaternary ammonium configuration. “Organosilane” also includes any quaternary ammonium salt of an organosilane. “Microbial cell” and “microbe” are used interchangeable and are understood to mean any single-celled organism. As the generally accepted size range for microparticles, a microcapsule has a size within the broad range of 1 micron to 1000 microns (1 millimeter). Therefore, the size range of a microcapsule, for the purposes of this application, is between that of a large nanoparticle to an object visible to the eye without magnification. Microparticle may also refer to a solid compound comprising the particle that is, itself, coated with the organosilane for purposes of becoming imbedded in a Conditioning Layer or more mature biofilm in an infected site.

Disclosed is an antimicrobial polymer product 100. Product 100 is an organosilane 102 in combination with other compounds in a mixture chosen according to the intended application of product 100.

The antimicrobial action of product 100 is provided by the organosilane. An organosilane is a molecule comprised of a silicone atom covalently bonded to carbon. Organosilanes in general may be amphiphilic, having both water-soluble and lipid soluble components. Organosilane 102 has a hydrophilic “cap” comprised of a silicon-tri-hydroxy “head,” and a hydrophobic “tail” comprising an eighteen or twenty-atom linear carbon chain. The head and tail are joined at a nitrogen atom bonded with two additional methyl groups to create a (cationic) quaternary ammonium group. The methoxy or hydroxy head groups facilitate enzymatically or chemically binding the organosilane to a surface 140. Surface 140 includes the non-limiting examples of biological surfaces, such as a dressing, filter or cotton cylinder for example. The hydrophilic quaternary ammonium group, in particular the positive charge of the Nitrogen atom, allows for ionic attraction between the negatively-charged molecular species unique to the external cell walls common to most bacteria and fungi. This difference in electrical charge that may compromise integrity of the microbial cell wall, plus entanglement of the silane's carbon chains that may pierce the cell wall of microbes is believed to be how death of microbes is caused. This microbial killing mechanism is advantageous for several reasons. Organosilane 102 is not altered or consumed by its interaction with the targeted microbe. The organosilane remains covalently bound to the treated material minimizing release of the compound into the environment.

In various embodiments of the invention, other compounds are added to product 100. For some embodiments a local anesthetic is added to reduce pain from inflammation. For some embodiments product 100 further comprises a cellulase enzyme. In some embodiments, product 100 further comprises other enzymes or compounds to interfere with quorum sensing utilized by microorganisms growing in a biofilm.

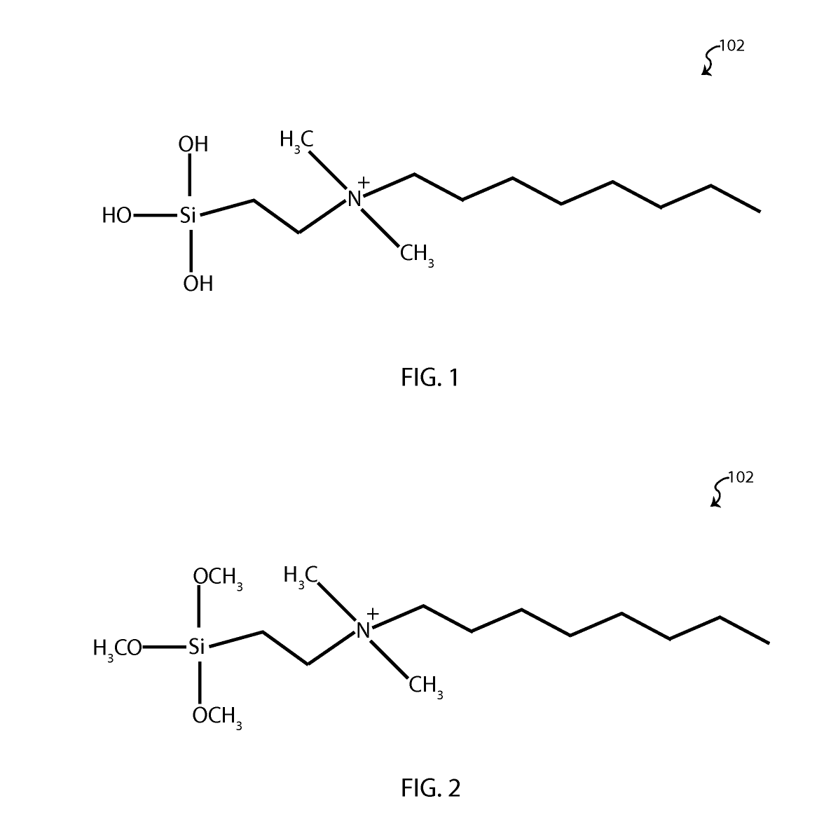

Referring to the drawings, FIG. 1 and FIG. 2 each depict an organosilane 102. These non-limiting examples show the fundamental structure of two organosilanes 102 with antimicrobial activity. Product 100, in some embodiments, may comprise an organosilane 102 with alternative molecular structures. Common to organosilanes 102, however, are a silyl “head,” a quaternary ammonium group, and an aliphatic hydrocarbon “tail.” Embodiments of product 100 comprise organosilane 102 and, in some embodiments, additional structural and functional components that complement one another to add functionality and performance to product 100, the structure and function of which will be described in greater detail herein.

In the example embodiment shown in FIG. 1, organosilane 102 is a 3-hydroxysilyl organosilane. The silyl “head” of the molecule is shown to the left of the figure, comprising three hydroxyl groups which, in some embodiments, are reacted to covalently bond with a biological or non-biological surface. The quaternary ammonium group is also shown, connecting the silyl “head” with the aliphatic hydrocarbon “tail.” In the example embodiment shown in FIG. 2, organosilane 102 is a 3-methoxysilyl organosilane. In some embodiments, organosilane 102 is s 3-(trihydroxysilyl) propyl dimethyl octadecyl ammonium molecule. In some embodiments, organosilane 102 is a 3-(trimethoxysilyl) propyl dimethyl octadecyl ammonium molecule. In some embodiments, organosilane 102 is s 3-(trihydroxysilyl) propyl dimethyl dodecyl ammonium molecule. In some embodiments, organosilane 102 is a 3-(trimethoxysilyl) propyl dimethyl dodecyl ammonium molecule. Electrostatic and Mechanical killing of microbial cells 135 occurs by penetration and physical disruption of the microbial cell wall and phospholipid cell membrane by the hydrophobic “tail” of organosilane 102. Microbial cells 135 are drawn to a treated surface 140 covered with adherent organosilane 102 molecules electrostatically by the cationic quaternary ammonium groups of organosilane 102. Amphiphilic quaternary ammonium compounds, including but not limited to organosilane 102, may effect microbial killing by acting as detergents, wherein the cationic N⁺ atom ionically binds to negatively charged sites on lipopolysaccharides and constituent proteins of the bacterial cell wall, bringing the hydrophobic organosilane “tail” into proximity with the phospholipid cell membrane. In some embodiments, addition of other detergents to product 100 facilitate penetration of organosilanes 102 into subsurface layers of microbial biofilms by disrupting the complex mixed hydrophilic/hydrophobic layers of the biofilm. Some non-limiting examples of detergents added to product 100, in some embodiments, include other quaternary ammonium compounds, benzalkonium chloride, benzethonium chloride and sodium oxychlorosene.

Some antibiotics and enzymes function optimally within a relatively narrow pH range. Accordingly, some embodiments of product 100 add a buffer to the treating compound at concentration levels sufficient to maintain the pH range required for optimal activity of the components of the product. The particular buffer is selected based upon the local conditions present on the biological surface 140. Buffers to maintain ambient pH within a desired range include, but are not limited to, citrates, sulfonates, carbonates, and phosphates. The preferred buffering compound and concentration of same useful for maintaining a desired pH range are dependent on ambient micro-environmental conditions at the treated area and known to those skilled in the art.

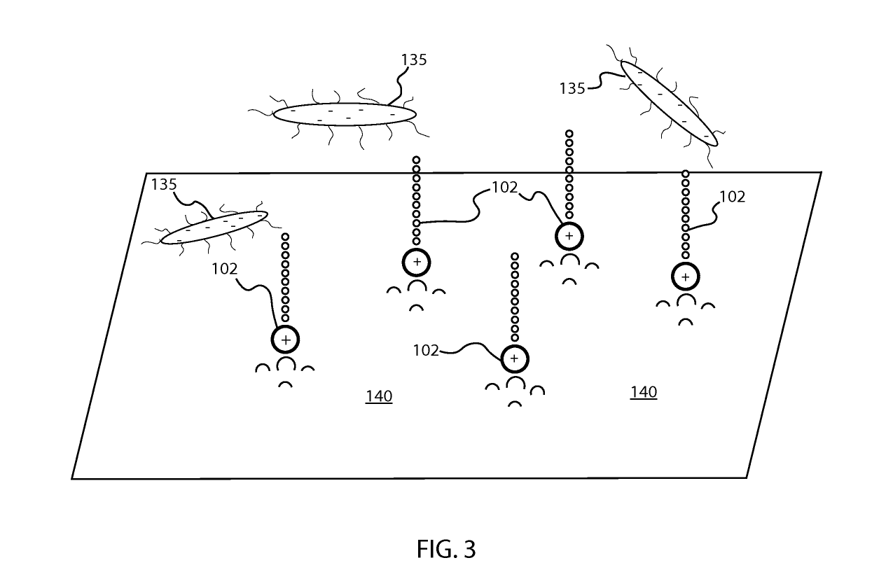

FIG. 3 is a diagram showing organosilane 102 molecules bonded to a surface 140 in the presence of a microbial cell 135. Microbial cells 135 may be bacteria (as shown in FIG. 3), archaebacteria, protists, or fungi. A microbe generally carries a negative net charge at the cellular surface due to constituent membrane proteins. For example, the cell walls of Gram-positive bacteria contain negatively-charged teichoic acids. The cell membranes of Gram-negative and Gram-positive bacteria (and other microbes) comprise negatively charged phospholipids and lipopolysaccharide molecules. The negatively-charged surfaces of free-floating “planktonic” microbes, therefore, are electrostatically attracted to cationic compounds, such as the quaternary ammonium group-containing organosilane 102 coating surface 140. The difference in ionic charge may cause adherence to the cell contributing to the disruption of the cell wall and cytoplasmic membrane. If the compound, such as organosilane 102, is amphiphilic, the hydrophilic portion of the molecule may traverse both the bacterial cell wall and cytoplasmic membrane, causing cellular lysis and death of microbial cell 135. As a result, the attachment and bonding of product 100 comprising organosilane 102 to surface 140 results in surface 140 becoming configured to kill microbial cells 135 on contact. Because this surface killing does not disrupt and consume product 100, frequently repeated application is not required and as a result, cell destruction is accomplished without releasing a biocide to the environment.

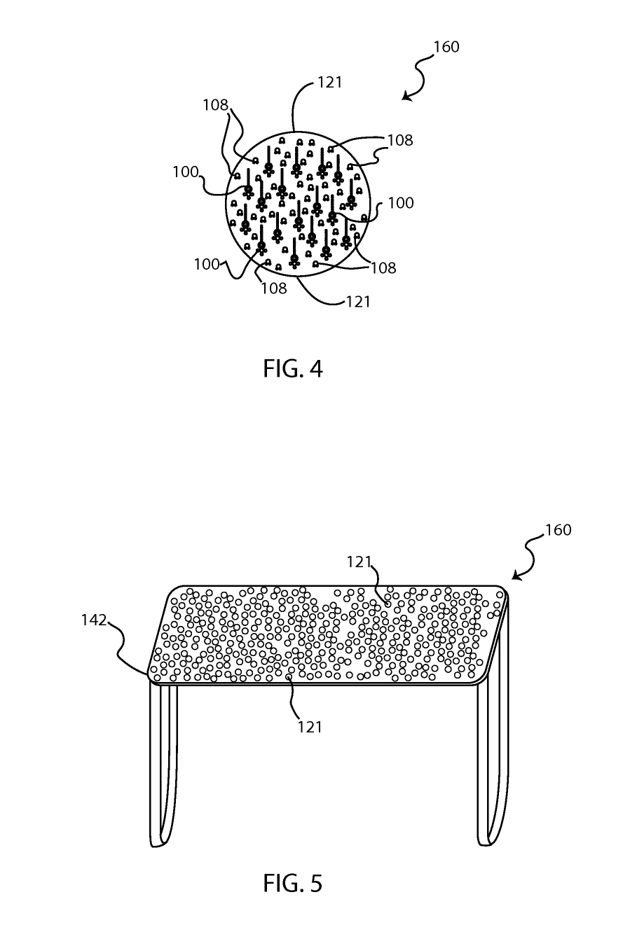

Product 100 additionally comprises a carrier 108, schematically shown in FIG. 4. Carrier 108, in some embodiments, is a compound that holds the various sub-components of product 100 in suspension or solution. The specific compound used is chosen based upon the characteristics necessary for the end-use application of product 100. In some embodiments, carrier 108 comprises compounds carried in microcapsules or on nano particles and used to incorporate organosilanes, enzymes and additional antimicrobials.

The concentration of organosilane 102 by weight of product 100 is also selected according to the desired end-use of product 100. In situations where high antimicrobial activity is needed for treating a non-biological surface, higher concentrations of organosilane provide a higher density of adherent organosilane molecules on surface 140. In effect, the “forest” of aliphatic hydrocarbon molecular “tails” is thicker. Additionally, higher organosilane concentrations create a higher cationic charge density, resulting in both stronger electrostatic microbial attractive forces and detergent effects on the microbial phospholipid cell membrane. Concentrations of organosilane 102 in product 100 of up to and over 5% by weight may be used, however, when used in concentrations of over about 3%, polymerization of organosilane 102 within product 100 prior to application on surface 140 increases through intermolecular cross-linking via —S—O—S— covalent bonds. The risk of bacterial and other microbial resistance to an antimicrobial compound, regardless of the mechanism of action of the compound, theoretically increases with increasing environmental encounters between bacteria and other microbes, and the antimicrobial compound. It is prudent, therefore, to strive to minimize the amount of an antimicrobial compound within the general environment. Accordingly, in the aforementioned and other situations wherein frequent re-application of product 100 is necessary, lower concentrations of organosilane 102, down to and below 0.1% by weight in product 100, are useful by lowering the overall amount of organosilane 102 ultimately discharged into the environment. Notwithstanding the theory, it is believed that the risk to the environment and/or causing microorganism mutations by use of these formulations is minimal.

Because in some embodiments, product is a non-leaching compound that is bound to surface 140 of an article 142, the area may be treated without ever placing or applying the antimicrobial product directly into the area 24 or in physical continuity with the area 24, if desired

Advantages of product 100 according to the several embodiments described herein, include the aforementioned non-leaching properties of product 100, decreased risk of biofilm resistance given the unique mechanical disruption of cell walls and cell membranes common to all microbes, and the relative non-toxicity, the stability of product 100 bonded to surface 140 safely providing for subsequent dispersal of organosilanes and other constituent compounds of product 100 into the environment. The relative non-toxicity of organosilanes when used to filter and kill air born pathogens and treat surface infection using dressings and cotton cylinders.

In view of the aforementioned limitations, some embodiments of product 100 further comprise a delivery system 160, as shown in FIG. 4 and FIG. 5. The delivery system 160 be delays activation of product 100 to some point after application upon filter, guaze or the like of 140, when the antimicrobial action of product 100 is needed. Thus, delivery system 160 “schedules” the useful life-span of product 100's antimicrobial activity within a timeframe controlled by the end user based on when antimicrobial activity is most needed. When antimicrobial activity decreases, the thin-layer of product 100 and any associated, entangled cellular debris is removed from the surface prior to re-application of fresh product 100. In addition to proteolytic keratinases, some embodiments of product 100 comprise other enzymes. For example, N-acyl homoserine lactone is a bacterially-produced amino sugar acting as a hormone involved in quorum sensing, wherein a population of bacteria limits its growth density and other population-based characteristics, such as gene regulation of enzyme systems and the expression of flagella versus pili.

FIG. 4 shows a microcapsule 121 encasing product 100. FIG. 5 shows an article 142 embedded with microcapsules 121. In the example shown in FIG. 5, article 142 is part of a face mask. This is in no way meant to be limiting. Article 142, in some embodiments, is an object, material, article, thing, or body manufactured and treated in such a way that the microcapsules 121 are preserved until the user desires to dispense, distribute, utilize, apply, operate, activate, or otherwise disinfect or utilize article 142. In some embodiments, article 142 is non-disposable. In some embodiments, article 142 is disposable. In some embodiments, article 142 is manufactured comprising delivery system 121 containing product 100. In some embodiments, delivery system 121 is applied to article 142 by the end-user following manufacture of article 142. In some embodiments, delivery system 121 containing product 100 is applied to article 142 by the end-user following manufacture of article 142.

Microcapsule 121 is one example of delivery system for product 100. Microcapsule 121, in some embodiments, comprises a material enveloping and containing a core of product 100. Non-limiting examples of which include polyvinyl alcohol, cellulose acetate phthalate, gelatin, ethyl cellulose, glyceryl monostearate, bee's wax, stearyl alcohol, and styrene maleic anhydride. Many other compositions of microcapsule 121 are possible, and the exact composition, construction, and manufacture of microcapsule 121 is chosen from the broad range of compositions and manufacturing techniques for microcapsules generally, and which are readily available and known to those skilled in the art. In the example delivery system shown in FIG. 4 and FIG. 5, liquid product 100 is encapsulated within microcapsule 121 and thereafter released when microcapsule 121 is broken. Breakage of microcapsule 121 is effected at a chosen time and in a manner specific to the particular use of product 100. For example, microcapsule 121 may be manufactured and/or embedded in article 142, such as for example, but not in any way limiting, an article 142. Non-limiting examples of article 142 include medical devices, disposable medical devices, gauze dressings, other dressings, wound coverings, implantable medical devices, and the like. At a time and under any condition selected by the end-use, upon breaking the microcapsule 121 the liquid form of product 100 may be adsorbed onto surface 140 of article 142, in some embodiments permeating the material composition of surface 140 and thereby creating article 142 treated with persistent, long-acting surface disinfectant properties. In this manner, in some embodiments, product 100 is configured to remain on article 142. Because product 100 becomes active upon breaking of microcapsule 121, the effective useful life of product 100 begins at the time desired by the user. Thus, the use of delivery system 160 comprising microcapsule 121 to distribute and apply product 100 to surface 140 will ensure that the product 100 is freshly applied and biologically active when the user desires and within the user's timeframe. In the case of a smaller article 142, such as a disposable surgical instrument for example, embodiments of delivery system 160 may comprise breaking, piercing, or puncturing microcapsules 121 to release product 100 upon the removal of article 142 from packaging material. Non-limiting examples may comprise activation (i.e., breaking) of microcapsules 121 on articles 142 to release product 100 as disposable articles 142 are removed from packaging.

Some embodiments of delivery system 160 for product 100 require application of a direct force to the treated surface 140 by the user. In some non-limiting examples, product 100 is applied to a disposable surgical facemask, encapsulated in the delivery system 160 of microcapsules 121 embedded in the facemask. In particular, delivery system 160 comprising microcapsules 121 is applied to a sanitized, middle layer of a 3-layered facemask prior to use of the facemask. Once the user desires to use the new facemask, however, the user must apply force to the facemask, for example by folding and rubbing the sides together to break microcapsules 121 to deliver product 100 to the middle layer of the facemask. Once released from broken microcapsules 121, product 100 dries and thereafter functions as a microscopic bed of product 100 that effectively destroys microbes which encounter the middle layer of the facemask via air currents created by the wearer's breathing. As described herein, delivery system 160, may be utilized with any textile, fabric, cloth, material, object, entity, and/or thing, whether porous or non-porous, to timely deliver product 100 thereto for effective use as an antimicrobial agent.

Embodiments of the delivery system 160 of product 100 may further comprise application of microcapsules 121 encasing product 100 into medical devices and/or medical supplies. In particular, product 100 may be applied via microcapsules 121 to surfaces 140 of bandages, gauze, gauze dressings, sutures, cotton balls, cotton dressings, cotton swabs, cotton tipped applicators, cotton rolls, and other similarly-purposed medical supplies.

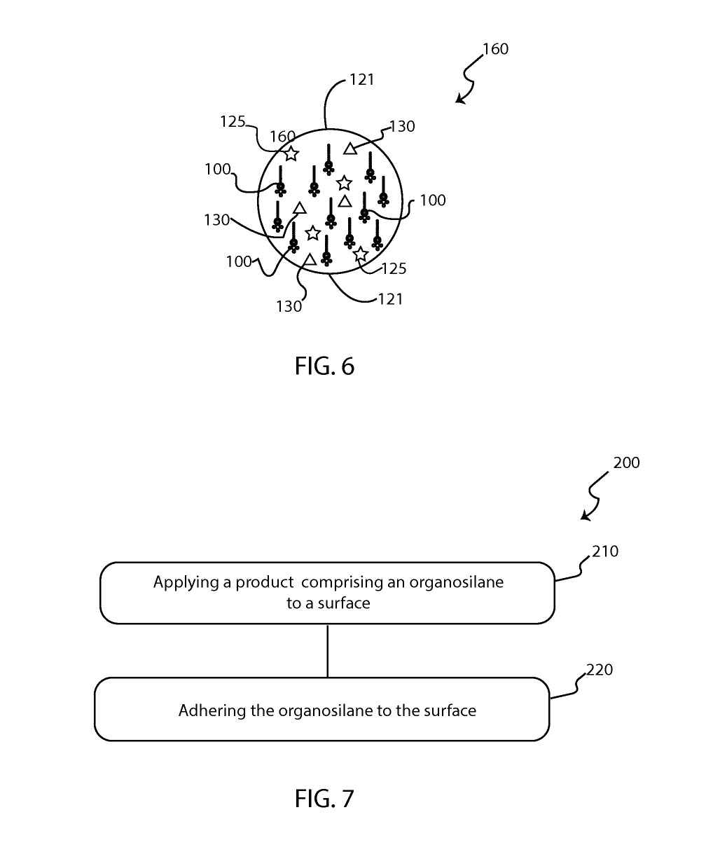

FIG. 6 is a schematic representation of delivery system 160 comprising microcapsule 121 enclosing product 100, aging indicator 125, and fragrance 130. Medical treatments and/or medical supplies bearing delivery system 160 with product 100 embedded therein by microcapsules 121 for later release and delivery of product 100 to the surfaces 140 at a time desired by the user may also comprise a fragrance 130 and/or an aging indicator 125. Force is applied by the user to article 142 treated with delivery system 160 comprising microcapsules 121 sufficient to break microcapsules 121 and release product 100 on surfaces 140 of article 142. After released product 100 has bonded to surface 140, article 142 may be utilized in the prevention and/or treatment of infection and infectious disease, as herein described.

Embodiments of the delivery system 160, in some embodiments, further comprise fragrance 130, as shown in FIG. 6. Fragrance 130 may be encapsulated within microcapsule 121 along with product 100. Fragrance 130 may also be bonded to the exterior surface of microcapsule 121 and configured to react with product 100, upon release of product 100 following rupture of microcapsule 121. Fragrance 130, in some embodiments, is configured to exert, exhibit, or otherwise create a detectable scent for a predetermined period of time following activation of fragrance 130. Embodiments of delivery system 160 may comprise fragrance 130 bearing a scent that persists as long as product 100 remains biologically active. In other words, some embodiments of delivery system 160 comprise a set length of time calculated and configured to match the expected biologically active life of product 100. The biologically active life of product 100 may be accurately determined based upon factors such as the characteristics of substrate 140, external environmental conditions, and concentration and amount of product 100 used, for example. In this manner, the user is able to determine by the presence of the scent emanating from fragrance 130 whether product 100 remains biologically active or has expired. Once expired, the user may determine by the lack of a scent that product 100 on is no longer biologically active and that consideration should be given to sterilize and re-treat or to discard article 142.

As also shown in FIG. 6, some embodiments of delivery system 160 for product 100 further comprise aging indicator 125. Aging indicator 125, in some embodiments, is encapsulated in microcapsule 121 along with product 100. Aging indicator 125, in some embodiments, is bonded to the exterior surface of microcapsule 121 and configured to activate a coloring agent or fading agent upon contact with product 100. Aging indicator 125, in some embodiments, is configured to exert, exhibit, or otherwise release a color, fading agent, or time-dependent color that changes color over a predetermined period of time after aging indicator 125 has been activated. Some embodiments of delivery system 160 comprise aging indicator 125 comprising a fading color or time-dependent color that changes color or alters color for a time period matching the useful life of product 100's biological activity. In other words, some embodiments of the delivery system 160 comprise a time period calculated and configured to match the anticipated life expectancy of product 100. The biologically active life of product 100 may be determined based upon factors such as the characteristics of surface 140, external environmental conditions, concentration, and amount of product 100 used, for example. In this way, the user is able to determine by the color, faded color, or time-dependent color whether product 100 remains biologically active or has expired. Once expired, the user may determine by the lack of a scent that product 100 on is no longer biologically active and that consideration should be given to re-treat or discard article 142.

FIG. 7 shows a method 200 of treating infection and/or infectious disease and/or providing long-lasting disinfectant properties to a substrate Method 200 comprises an applying step 210 and an adhering step 220. Step 210 of method 200 comprises applying product 100 comprising an organosilane to a substrate. Step 210, in some embodiments, includes applying product 100 to a supply or article. Step 220 of method 200 comprises adhering the organosilane to the substrate. Adhering step 220 occurs when the “head” group of the organosilane, such as hydroxy or methoxy groups for example, reacts with the material composition of the treated substrate to adhere to the surface. In some embodiments, adhering step 220 comprises formation of covalent bonds between the organosilane and the surface. In some embodiments, adhering step 220 comprises adsorption onto a non-porous substrate or into a porous surface. In some embodiments, adhering step 220 comprises an electrostatic interaction between the organosilane and the substrate, such as formation of ionic chemical bonds, for example. In some embodiments, adhering step 220 comprises addition of an additional compound, such as a catalyst, to accelerate reaction of the organosilane with the material of the treated article. In some embodiments, other bonding agents and/or techniques are employed to facilitate bonding of product 100 with the material of the treated article.

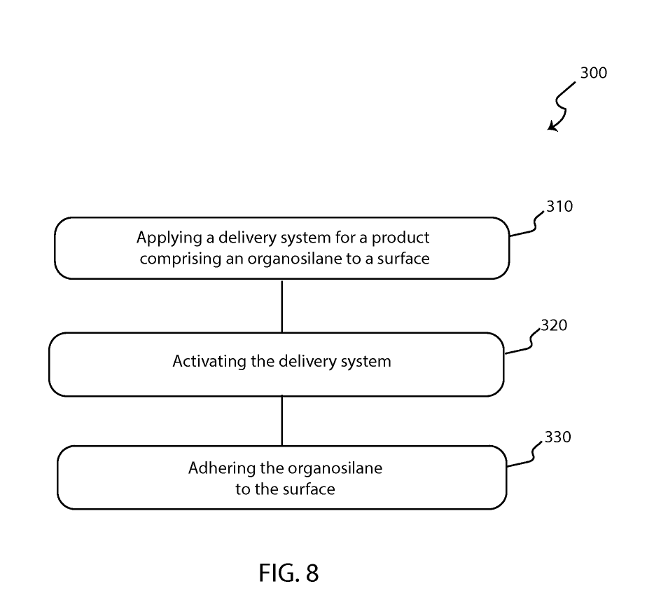

FIG. 8 shows a method of treating infection and/or infectious disease and/or providing short term disinfectant properties to a surface. Method 300 comprises an applying step 310, an activating step 320, and an adhering step 330. Step 310 of method 300 comprises applying a delivery system for a product comprising an organosilane to a surface. In some embodiments, the delivery system of applying step 310 comprises a microcapsular delivery system, such as microcapsule 121 discussed herein above. In some embodiments, the delivery system of applying step 310 is a delivery system not comprising microcapsules. In some embodiments, applying step 310 comprises integrating the delivery system, such as by imbedding microcapsules containing a product comprising an organosilane for example, into part or all of the material composition of the treated article during manufacture. For example, the felt cuff encircling a portion of a trans-cutaneous central venous catheter, in some embodiments, is manufactured to contain a quantity of microcapsules upon and intermingled within the fibers throughout the felt cuff. Step 320 of method 300 comprises activating the delivery system. In some embodiments, activating step 320 comprises breaking microcapsules encasing the product comprising the organosilane. An additional non-limiting example of activating step 320, in some embodiments, includes removing the treated article from its packaging. In the central venous catheter example mentioned earlier, activating step 320 may, in some embodiments, comprise passing the segment of the catheter bearing the treated felt cuff tangentially through the skin insertion/puncture site wherein microcapsules encasing product are mechanically ruptured as the cuff passes through the skin. Adhering step 330 of method 300 comprises adhering the organosilane to the substrate. In some embodiments, such as the aforementioned central venous catheter example, adhering step 330 involves reaction of released, activated components of the product, including the organosilane, for example, bonding to the material of the felt cuff, the surface of the catheter, and proteins located within the intercellular matrix of the subcutaneous tissue. Bonding of the organosilane to the surface may be by covalent bonding, ionic bonding, electrostatic bonding, or other interaction between the organosilane and the surface material.

Method 200 and/or method 300 may further comprise placing the one or more treated articles into a location prone to microbial growth, such as an area where microbes are known or expected to exist, and/or an area 24 where biofilms have formed or may develop. Alternatively, the treated article may be placed into close proximity to such an area but not directly in the area. By placing the treated article into close proximity but not directly into contact with such an area, the electrostatic properties of product 100 comprising an organosilane or and/or additional cationic detergent or other substance may attract and draw nearby microbes to the cationic product, thereby reducing the concentration of microbes in the adjacent area sought to be protected from microbial colonization and/or infection and possible biofilm formation.

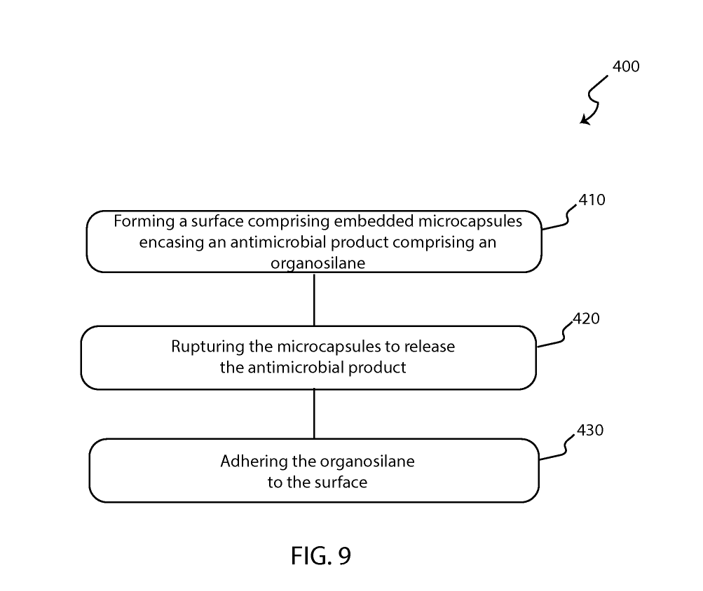

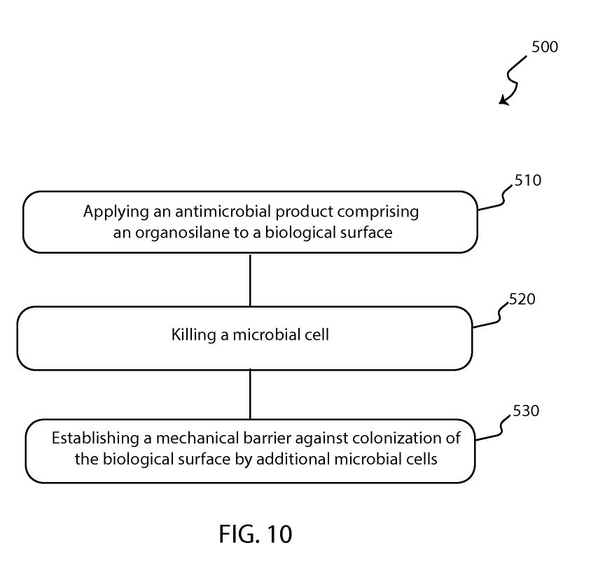

FIG. 9 shows a method 400 of forming an article with an antimicrobial product delivery system embedded therein. Method 400 comprises a forming step 410, a rupturing step 420 and an adhering step 430. Forming step 410, in come embodiments, comprises incorporating a microcapsular delivery system of an antimicrobial product comprising an organosilane into the material composition of an article during manufacture of the article. Rupturing step 420 comprises rupturing of the microcapsules by physical/mechanical, chemical, or electrical means, thereby effecting the release of the antimicrobial product from the delivery system. Adhering step 430 comprises adherence of the organosilane molecules to the surface, such as by covalent bonds, ionic bonds, electrostatic forces, or other molecular interactions between the organosilane and the material composition of the treated surface.

The disclosed product minimizes leaching of antimicrobial into the environment, minimizes opportunities for development of microbial resistance due to its combined mechanical and electrostatic mechanisms of action, is safe and effective for use to filter air borne pathogens to and from the respitaory system, in dressings to aid in treating infections and may be incorporated directly onto articles such as medical devices and supplies by way of a delivery system.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

What is claimed is:

1. An antimicrobial product comprising:
an organosilane;
a carrier;
a delivery system; and
a facemask, wherein the delivery system delivers the organosilane and carrier to the facemask.

2. The antimicrobial product of claim 1, wherein the delivery system is a microcapsule enclosing the antimicrobial product therein.

3. The antimicrobial product of claim 2, further comprising a fragrance, wherein under a condition wherein the microcapsule is broken, the antimicrobial product is adhered onto the facemask and the fragrance becomes active; and wherein a time of useful antimicrobial activity of the activated antimicrobial product corresponds to a time wherein a scent of the active fragrance is present.

4. The antimicrobial product of claim 1, wherein the organosilane is a 3-(trimethoxysilyltrihydroxysilyl) quaternary ammonium compound or a 3-(trimethoxysilyl) quaternary ammonium compound.

5. The antimicrobial product of claim 1, wherein the concentration of the organosilane is less than 0.10 percent by weight.

6. The antimicrobial product of claim 1, wherein the concentration of the organosilane is between 0.10 percent and 1.00 percent by weight.

7. The antimicrobial product of claim 1, wherein the concentration of the organosilane is greater than 1.00 percent by weight.

8. The antimicrobial product of claim 1, wherein the concentration of the organosilane is greater than 5.0 percent by weight.

9. The antimicrobial product of claim 1, wherein the carrier is a compound selected from the group of carrier compounds consisting of: an alcohol, a wax, or dimethylsulfoxide.

10. The antimicrobial product of claim 1, further comprising an enzyme.

11. The antimicrobial product of claim 10, wherein the enzyme is a proteolytic keritinase enzyme.

12. The antimicrobial product of claim 10, wherein the enzyme is an enzyme acting upon a substrate comprising N-acyl homoserine lactone.

13. The antimicrobial product of claim 1, further comprising a detergent.

14. The antimicrobial product of claim 12, wherein the detergent is a quaternary ammonium compound.

15. The antimicrobial product of claim 1, wherein the antimicrobial product further comprises an antiseptic.

16. The antimicrobial product of claim 15, wherein the antiseptic is a compound selected from the group of antiseptic compounds consisting of: benzethonium chloride, benzalkonium chloride, sodium oxychlorosene, hypochlorous acid, hexylresorcinol, methyl resorcinol, poloxamer iodine complex, iodine complex, secondary amyltricresols, and ethyl alcohol.

17. The antimicrobial product of claim 1, wherein the antimicrobial product further comprises a disinfectant.

18. The antimicrobial product of claim 2, wherein the antimicrobial product further comprises a buffer.

19. The antimicrobial product of claim 18, wherein the buffer is a compound selected from the group of buffer compounds consisting of: a citrate, a sulfonate, a carbonate, and a phosphate.

20. A method of providing an antimicrobial treatment to a surface, the method comprising the steps of:
embedding microcapsules encasing an antimicriobial product comprising an organosilane in a porous or filter material;
activating the delivery system; and
adhering the material to the area of infection to be treated.

21. The method of claim 20, further comprising a step placing the surface in proximity to an area of infection.

22. A method of treating an infection, the method comprising steps:
applying an antimicrobial product to a biological surface;
killing pathogens; and
establishing a mechanical barrier against re-infection of the biological surface by additional microbial cells.
